(19) United States
(12) Patent Application Publication
(10) Pub. No.: US 2022/0354343 A1
(43) Pub. Date: Nov. 10, 2022

(54) MEDICAL DEVICE SYSTEMS

(71) Applicant: Boston Scientific Scimed, Inc., Maple Grove, MN (US)

(72) Inventors: Scott BRECHBIEL, Acton, MA (US); James WELDON, Newton, MA (US)

(73) Assignee: Boston Scientific Scimed, Inc., Maple Grove, MN (US)

(21) Appl. No.: 17/662,004

(22) Filed: May 4, 2022

Related U.S. Application Data
(60) Provisional application No. 63/184,266, filed on May 5, 2021.

(51) Int. Cl. A61B 1/005; A61M 25/01

(52) U.S. Cl. CPC A61B 1/0052; A61M 25/0136; A61M 2205/586

(57) ABSTRACT

A medical system comprising a first device comprising a first handle and a first shaft extending distally from the first handle, wherein the first handle includes a first actuator, a second device comprising a second handle and a second shaft extending distally from the second handle, wherein the second handle includes a second actuator, and a fixture coupling the first device to the second device, wherein a center of the first actuator is within 4.5 inches of a center of the second actuator, and wherein the fixture couples the first device to the second device so that the first handle is configured to be held in a first hand of an operator, while a second hand of the operator traverses between use of the first actuator and the second actuator.

### CROSS-REFERENCE TO RELATED APPLICATION(S)

This application claims the benefit of priority from U.S. Provisional Application No. 63/184,266, filed on May 5, 2021, which is incorporated by reference herein in its entirety.

### TECHNICAL FIELD

This disclosure relates generally to medical device systems. More particularly, at least some embodiments of the disclosure relate to systems including multiple medical devices ergonomically positioned relative to one another.

### BACKGROUND

In some medical procedures, e.g., endoscopic procedures, a first device, such as a duodenoscope, and a second device, such as a catheter, may be used concurrently. Both the first device and the second device may each have a knob configured to control an aspect/feature of the device. Thus, in certain instances, physicians must operate two sets of knobs, an insertion tube of said second device, and an accessory device inserted into the second device, while also maintaining the position of the first device relative to the targeted site. To do so, in situations involving a scope and a catheter, physicians generally maintain scope articulation/position with the left hand while controlling the catheter position and advancement with the right hand. Because the catheter is generally mounted to a distal end of the handle of the first device, physicians are required to constantly translate their right forearm and wrist along a parallel knob stack, which may not be ergonomic.

### SUMMARY OF THE DISCLOSURE

According to an example, a medical system may comprise a first device comprising a first handle and a first shaft extending distally from the first handle, wherein the first handle includes a first actuator, a second device comprising a second handle and a second shaft extending distally from the second handle, wherein the second handle includes a second actuator, and a fixture coupling the first device to the second device, wherein a center of the first actuator is within 4.5 inches of a center of the second actuator, and wherein the fixture couples the first device to the second device so that the first handle is configured to be held in a first hand of an operator, while a second hand of the operator traverses between use of the first actuator and the second actuator.

In another example, during use, the second shaft may enter a port of a handle of the first device, the first device may be configured to be closer to the operator than the second device, and the first actuator may be configured to be below the second actuator.

In another example, during use, the second shaft may enter a port of a handle of the first device, the first device may be configured to be further from the operator than the second device, and the first actuator may be configured to be above the second actuator.

In another example, the first actuator may lie on a first plane, the second actuator may lie on a second plane, and the second plane may be at an angle relative to said first plane, wherein the angle between the second plane and the first plane is between 5° and 70°. The first actuator and the second actuator may also lie on a shared plane or parallel planes.

In another example, the first actuator may include a first rotatable knob, the second actuator may include a second rotatable knob, and an axis of rotation of the first rotatable knob may be within 2.75 inches of an axis of rotation of the second rotatable knob.

In another example, the coupling fixture may comprise a first section receiving the first device and a second section receiving the second device, wherein the second section is configured to receive the second device at any angular orientation of the second device relative to the first device. The first section may receive a portion of the first handle of the first device, and the second section may receive a portion of the second handle of the second device. The first section may include a first channel configured to receive the portion of the first handle, the second section may include a second channel configured to receive the portion of the second handle, and the coupling fixture may further include an arm extending between the first section and the second section. The first device may further comprise a connector cable extending proximally, wherein the first section receives a portion of said connector cable, and the second section receives a portion of the second handle of the second device. The coupling fixture may further comprise an adjustable arm extending between the first section and the second section, wherein the adjustable arm is configured to be maneuverable so that the second section is at any angular orientation relative the first section.

In another example, the first device may further comprise a first port, the second device may further comprise a second port and a third port, and a portion of the second device may be coupled to the first port. The second shaft of the second device may be configured to extend from a proximal end at the second port to the third port, and the third port of the second device may be in fluid communication with the first port of the first device.

In another example, the second device may be configured to be oriented relative to the first device so that the second device can rest again the first hand when the first hand holds the first handle of the first device. The second device may be configured to be oriented relative to the first device so that the operator can access the first actuator and the second actuator via only forearm and/or wrist rotation.

According to another example, a medical system may comprise a first device comprising a first handle and a first shaft extending distally from the first handle, wherein the first handle includes a first actuator, a second device comprising a second handle and a second shaft extending distally from the second handle, wherein the second handle includes a second actuator, and a fixture coupling the first device to the second device, wherein the fixture couples the first device to the second device so that the first handle is configured to be held in a first hand of an operator, while a second hand of the operator traverses between use of the first actuator and the second actuator, and wherein a position of the second handle relative to a position the first handle is adjustable via the fixture to minimize the traversal of the second hand between use of the first actuator and the second actuator.

According to another example, a method of handling a medical system including a first device and a second device may comprise orienting the medical system so that the first device is closer to an operator than the second device, holding a handle of the first device with a first hand of the operator, and traversing between an actuator of the first device and an actuator of the second device with a second hand, via only wrist and/or forearm rotation of a second hand of the operator. The method may further comprise adjusting an angle of a second plane on which the second actuator lies relative to a first plane on which the first actuator lies, wherein the angle ranges from 5° to 70°. The medical system may further comprise a coupling fixture coupling the first device and the second device, and the angle is adjusted via adjusting the coupling fixture. The actuator may be a first rotatable knob, the second actuator may be a second rotatable knob, and an axis of rotation of the first rotatable knob may be within 2.75 inches of an axis of rotation of the second rotatable knob.

### BRIEF DESCRIPTION OF THE DRAWINGS

The accompanying drawings, which are incorporated in and constitute a part of this specification, illustrate various exemplary embodiments and together with the description, serve to explain the principles of the disclosed embodiments.

FIG. 1 is a perspective view of a medical system, according to an embodiment.

FIGS. 2A-2B are perspective views of a medical system, according to an embodiment.

FIGS. 2C-2D are perspective views of the medical system of FIG. 2A.

FIGS. 3A-3B are perspective views of a medical system, according to another embodiment.

FIGS. 4A-4C are perspective views of a medical system, according to another embodiment.

FIG. 5 is a perspective view of a medical system, according to another embodiment.

### DETAILED DESCRIPTION

Reference will now be made in detail to aspects of the disclosure, examples of which are illustrated in the accompanying drawings. Wherever possible, the same or similar reference numbers will be used through the drawings to refer to the same or like parts. The term “further” refers to position within a medical system, where a distance between said position and an operator of the medical system is larger relative to other distances being determined. By contrast, the term “closer” refers to a position within a medical system, where a distance between said space or position and the operator of the medical system is smaller relative to other distances being determined. The term “above” will refer to a position within a medical system that is situated higher when in use, e.g., closer to a ceiling, relative to other described positions of the medical system. By contrast, the term “below” will refer to a position within a medical system that is situated lower when in use, e.g., closer to the ground, relative to other described positions of the medical system. The term “distal” refers to a location or portion of a medical device farthest away from an operator of the device, e.g., when introducing a device into a subject (e.g., patient). By contrast, the term “proximal” refers to a location or portion closest to the operator, e.g., when placing the device into the subject. The term “upper” will refer to a location or portion of a medical device that is situated above other described portions of the medical device when the medical device is in an upright position, e.g., when a “distal” end of the medical device is below/lower than a remainder of the device. By contrast, the term “lower” will refer to a location or portion of a medical device that is situated below other described portions of the medical device when the medical device is in an upright position, e.g., when a “distal” end of the medical device is below/lower than a remainder of the device.

Both the foregoing general description and the following detailed description are exemplary and explanatory only and are not restrictive of the features, as claimed. As used herein, the terms “comprises,” “comprising,” “having,” “including,” or other variations thereof, are intended to cover a non-exclusive inclusion such that a process, method, article, or apparatus that comprises a list of elements does not include only those elements, but may include other elements not expressly listed or inherent to such a process, method, article, or apparatus. In this disclosure, relative terms, such as, for example, “about,” “substantially,” “generally,” and “approximately” are used to indicate a possible variation of ±10% in a stated value or characteristic.

Embodiments of the disclosure may solve one or more of the limitations in the art. The scope of the disclosure, however, is defined by the attached claims and not the ability to solve a specific problem. The disclosure, in certain embodiments, is drawn to a medical system including a first medical device, a second medical device, and a fixture coupling the first and second medical devices. The first or second medical device is not particularly limited. Either medical device may be, as an example, any scope (e.g., bronchoscope, duodenoscope, endoscope, colonoscope, ureteroscope, etc.), catheter, tool, instrument, or the like, having a shaft/catheter that extends distally from a handle to a distal portion of the device.

The shaft is not particularly limited, and may be any suitable flexible shaft configured to traverse bodily lumens during a procedure. The shaft may include at least one lumen for receiving any number of additional devices, e.g., scopes, tools, instruments, cables, fluids, or the like.

Similarly, the handle is also not particularly limited, and may be any suitable medical device handle. The handle may include at least one aspect for actuating or controlling the medical device, e.g., a knob stack, any tools or devices associated with the medical device, and/or the fastening mechanism of the medical device. Moreover, the handle may include at least one port, e.g., a Y-port, in fluid communication with a working channel, e.g., a lumen of the shaft. The port may receive any suitable accessory device, which may extend distally throughout the shaft and towards the distal end of the medical device. The handle may further include an additional port connected to an umbilicus that serves any suitable purpose. For example, said umbilicus may be for introducing fluid, suction, and/or wiring for electronic components.

The first medical device and the second medical device may be the same or similar devices. For example, the first medical device may be a larger scope, e.g., a mother scope, and the second medical device may be a smaller scope, e.g., the daughter scope. At least a portion of a flexible shaft of the daughter scope may be inserted through any suitable device port in fluid communication with the working channel, e.g., a lumen, of the shaft of the mother scope. The daughter scope may extend distally through the working channel of the mother scope, and either or both scopes may include imaging to visualize various portions of the anatomy. In turn, any suitable accessory device may be inserted through any suitable device port in fluid communication with a lumen of the catheter of the daughter scope.

The first and second devices may be ergonomically positioned with respect to one another. For example, the first and second devices may be arranged or positioned such that the distances between various contact points of the first and second device are minimized. Thus, there would be a reduction in arm translation by a physician, thereby improving access and procedural efficiency. Said contact points of the medical system may include the actuating or controlling feature, e.g., a knob stack, of the first medical device, the port of the first medical device, the actuating or controlling feature, e.g., a knob stack, of the second medical device, and the port of the second medical device. The first and second devices may be maintained in an ergonomic configuration via a fixture coupling both devices.

The fixture coupling the first and second medical devices, e.g., the mother scope and daughter scope, is not particularly limited. For example, the fixture may be any suitable component configured to couple the first and second medical devices in a desired configuration. In some embodiments, the fixture may be a separate component configured to receive/couple to the first device via a first feature, and receive/couple to the second device via a second feature. Neither the first feature nor the second feature is particularly limited, and may be openings, channels, magnetized features, straps, and other connective means of the fixture. In other embodiments, the fixture may be a component that may be at least partially integrated to a handle of the first device and/or a handle of the second device. In some other embodiments, the fixture may be a component configured to receive/couple to the first or second device via a feature, while being at least partially integrated to a handle of the other device.

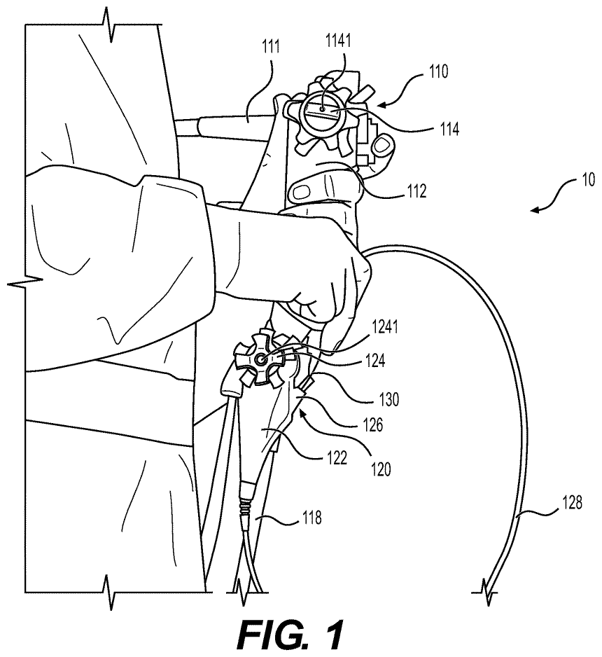

FIG. 1 illustrates an example of a typical medical system that is less ergonomically arranged. Medical system 10 includes a first medical device 110 and a second medical device 120. First medical device 110 includes a flexible shaft 118 (e.g., a catheter) and a handle 112 connected to a proximal end of shaft 118. Handle 112 is not particularly limited and may be any suitable handle configured for handling by an operator, e.g., a physician. Handle 112 includes an umbilicus 111, and an actuator, e.g., knobs, 114. Umbilicus 111 extends proximally relative to handle 112. Umbilicus 111 may be coupled to any suitable source, e.g., fluid, electrical, etc. Actuator 114 is coupled to an upper (proximal) portion of handle 112, and may be configured to control articulation of flexible shaft 118, and/or an articulation joint at a distal end of flexible shaft 118, in multiple directions. Actuator 114 may be, for example, one or more rotatable knobs that each rotates about its axis to push/pull actuating elements, e.g., steering wires (not shown). The actuating elements, such as cables or wires suitable for medical procedures (e.g., medical grade plastic or metal), extend distally from a proximal end of device 110 and connect to a distal portion of flexible shaft 118 to control movement thereof. Handle 112 further includes a port (not shown, but behind the operator's right hand, in FIG. 1) configured to receive a shaft 128 of second medical device 120 (described in further detail below).

Second medical device 120 may be similar to first medical device 110, as device 120 may include various similar components. Like first medical device 110, device 120 includes a flexible shaft 128, and a handle 122 connected to a proximal end of flexible shaft 128. As discussed above, shaft 128 is inserted into the port (not shown) of device 110, and extends distally throughout a working channel, e.g., a lumen of shaft 118 (not shown). Handle 122 includes an actuating device 124, which may also be one or more rotatable knobs that each rotates about its axis to push/pull actuating elements, e.g., steering wires (not shown), extending through device 120. Like device 110, device 120 may also include one or more electrical cables (not shown) providing electrical controls to imaging, lighting, and/or other electrical devices at the distal end of flexible shaft 128. Handle 122 also includes at least one port 126 for introducing and/or removing tools, fluids, or other materials from the patient. Port 126 may be used to introduce an accessory device (not shown), which may be any suitable tool or device for medical purposes.

As shown in FIG. 1, second device 120 is mounted or coupled to first device 110 via any suitable coupling means (not shown). As a result of such arrangement, actuator, e.g., knob stack, 114 of device 110, and actuator, e.g., knob stack, 124 of device 120 may lie on a parallel, or approximately parallel, plane. It is noted that in system 10, a distance between knob stack 114 and knob stack 124 may be larger, relative to other configurations discussed in further detail below. Furthermore, as can be seen in FIG. 1, system 10 allows for knob stack 124 and port 126 of device 120 to be accessible with one hand, e.g., the right hand, but limits availability of the other hand, e.g., the left hand, to assist with the handling of second device 120. In addition, system 10 may require an operator to move one hand, e.g., the left hand, distally on handle 112 to maintain stability while using device 120. Thus, by changing system 10 to one having a shorter proximity between the contact points—e.g., knob stack 114, knob stack 124, port of device 110 (not shown), and port 126 of device 120—there would be a reduction in travel from point to point while also reducing the need to remove one hand from its point of control.

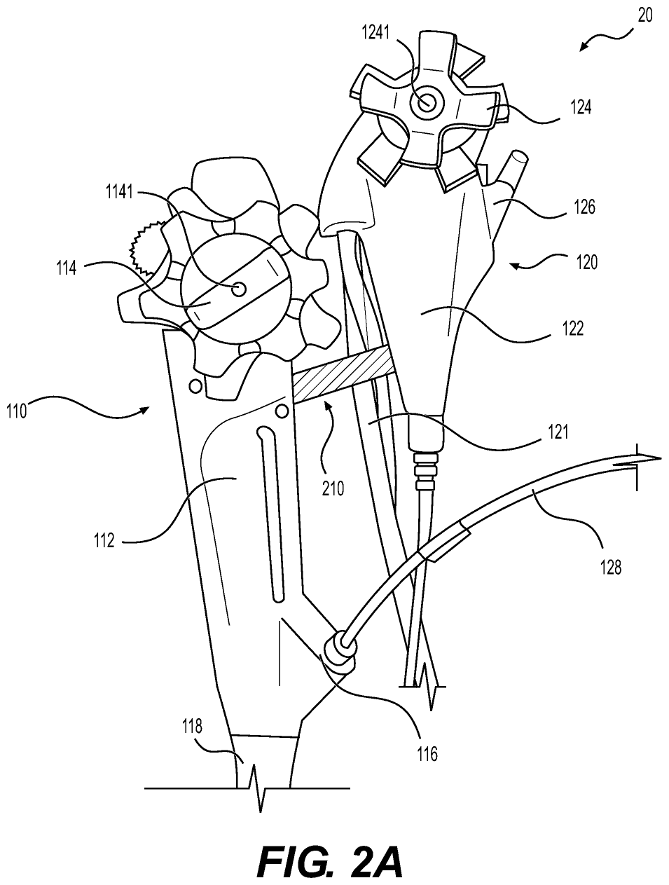

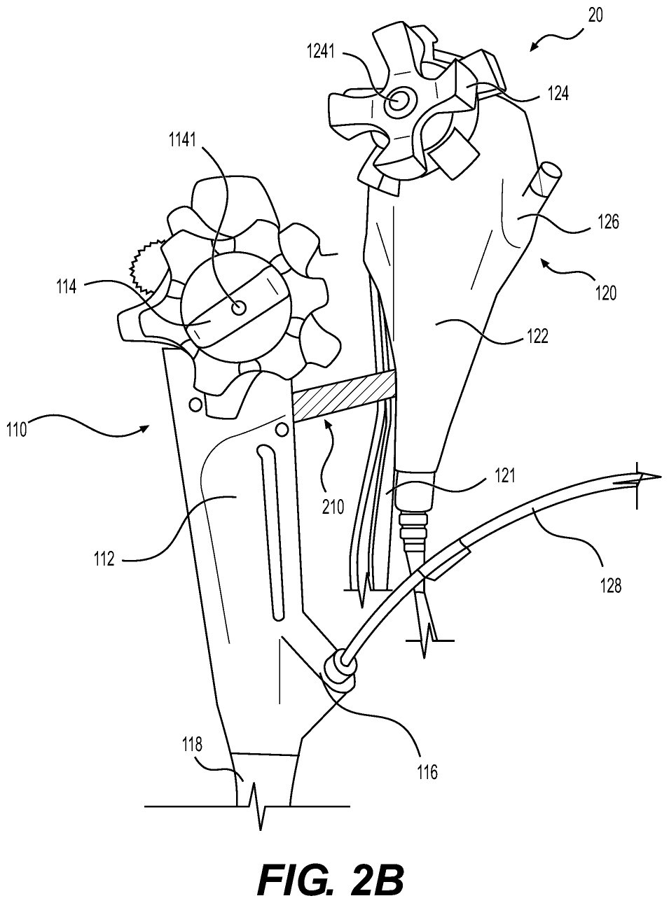

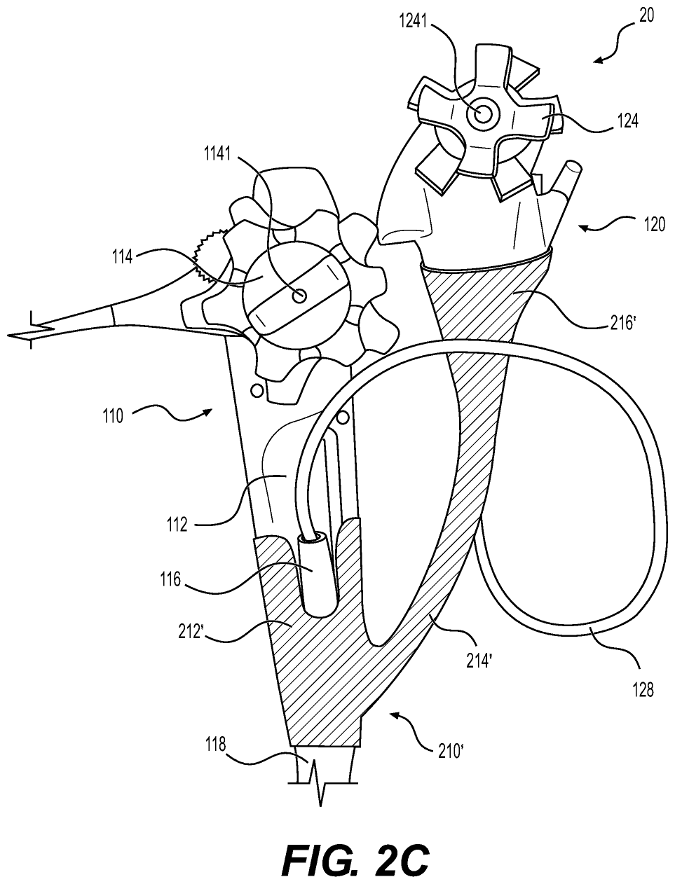

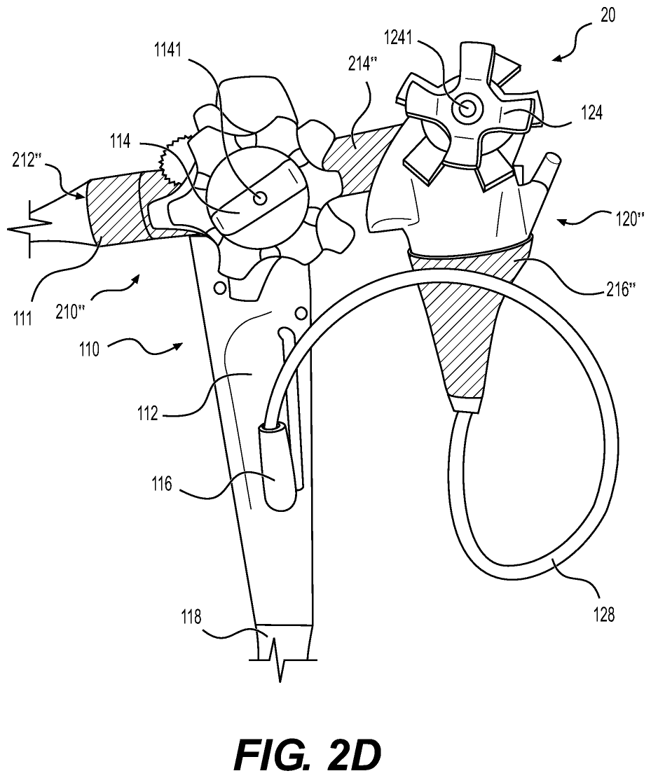

FIGS. 2A-2D illustrate an embodiment of a medical system 20 with improved ergonomics, relative to the configuration of FIG. 1. Like reference numerals refer to like parts. System 20 includes first medical device 110, second medical device 120, and a fixture 210. First medical device 110 and second medical device 120 may be the same devices as shown in system 10 of FIG. 1. Fixture 210 is not particularly limited, and may be any suitable coupling component configured to couple device 110 and device 120, and maintain both devices 110, 120 in the desired configuration. Further exemplary embodiments of fixtures 210′, 210″ are respectively shown in FIGS. 2C and 2D (discussed in further detail below).

Referring to FIG. 2C, fixture 210′ may include a first portion 212′, a linkage 214′, and a second portion 216′. First portion 212′ may be closer to an operator than second portion 216′. First portion 212′, at a first end of linkage 214′, includes a channel/slot configured to receive the distal or lower portion of handle 112 of first device 110. The dimensions of said channel/slot are not particularly limited. The manner in which handle 112 of first device 110 is secured into first potion 212′ is not particularly limited, e.g., frictional fit, clamping, locking, etc. Second portion 216′, at a second end of linkage 214′ opposite of portion 212′, includes a channel/slot configured to receive the distal or lower portion of handle 122 of second device 120. Similarly, the dimensions of said channel/slot of second portion 216′ are not particularly limited. The manner in which handle 122 of device 120 is secured into second portion 216′ is not particularly limited, e.g., frictional fit, clamping, locking, etc. Moreover, said channel/slot of second portion 216′ may be configured to receive handle 122 at any suitable angular orientation of the plane of knob stack 124 relative to other portions of device 110. Further discussion regarding such angular orientation of knob stack 124 is further detailed below, when describing FIG. 2B. Linkage 214′ may extend between first portion 212′ and second portion 216′, thereby coupling first device 110 and second device 120. The length which linkage 214′ may extend is not particularly limited, and may be any suitable length that may be functional and ergonomic for an operator. Linkage 214′ may be flexible or adjustable, e.g., including bellows, ball-socket, articulation links, etc., thereby allowing an operator to adjust the relative position of first medical device 110 or second medical device 120, with respect to the other. In addition, linkage 214′ should have sufficient structural integrity to retain its shape when an operator removes his/her hand (or other force) from any portion of system 20.

Referring to FIG. 2D, fixture 210″ is similar to fixture 210′ in various respects. First portion 212″ may be closer to an operator than second portion 216″. Fixture 210″ also may include a first portion 212″, a linkage 214″, and a second portion 216″. First portion 212″, at a first end of linkage 214″, includes a channel/slot configured to receive a portion of umbilicus 111 (or the strain relief of umbilicus 111) of first device 110. The dimensions of said channel/slot are not particularly limited. The manner in which umbilicus 111 or the strain relief is secured onto first portion 212″ is not particularly limited, e.g., frictional fit, clamping, locking, etc. Second portion 216″, at a second end of linkage 214″ opposite of portion 212″, includes a channel/slot configured to receive the distal or lower portion of handle 122 of second device 120. Similarly, the dimensions of said channel/slot of second portion 216″ are not particularly limited. Moreover, said channel/slot of second portion 216″ may be configured to receive handle 122 at any suitable angular orientation of the plane of knob stack 124 relative to other portions of device 110. Further discussion regarding such angular orientation of knob stack 124 is further detailed below, when describing FIG. 2B. The manner in which handle 122 of device 120 is secured into second portion 216″ is not particularly limited, e.g., frictional fit, clamping, locking, etc. Linkage 214″ may also extend between first portion 212″ and second portion 216″, thereby coupling first device 110 and second device 120. It is noted that in FIG. 2D, portions of linkage 214″ may be behind both handles 112 and 122. The length which linkage 214″ may extend is not particularly limited, and may be any suitable length that may be functional and ergonomic for an operator. Like linkage 214′, linkage 214″ may be flexible or adjustable, e.g., including bellows, ball-socket, articulation links, etc., thereby allowing an operator to adjust the relative position of first medical device 110 or second medical device 120, with respect to the other. In addition, linkage 214″ should have sufficient structural integrity to retain its shape when an operator removes his/her hand (or other force) from any portion of system 20.

System 20 yields improved ergonomics based on the positioning of first medical device 110 and second medical device 120, relative to one another. In system 20, first medical device 110 may be closer to an operator (shown in FIG. 1), and second medical device 120 may be further from the operator relative to device 110. Thus, in system 20, an operator may hold the closer, first device 110 with one hand, e.g., the left hand, and may utilize the other hand, e.g., the right hand, to traverse between and operate aspects of devices 110 and 120, e.g., knob stacks 114 and 124. Furthermore, second medical device 120 is also positioned such that knob stack 124 is slightly above knob stack 114 (as shown in FIGS. 2A-2D). When system 20 is in use, and devices 110 and 120 are in the positions shown in FIG. 2A (handles 112 and 122 essentially upright/vertical), the degree by which knob stack 124 is above knob stack 114 may be, for example, from 0° to less than 90° degrees, or 0° to 45° degrees between a horizontal line extending through a center point 1141 of knob stack 114 and another line extending from point 1141 and a center point 1241 of knob stack 124. Thus, the distance between knob stacks 114 and 124 in system 20 may be minimized relative to said distance in system 10. In some examples, the distance between center point 1241 of knob stack 124 and center point 1141 of knob stack 114, in system 20, may be less than or equal to 6 inches, 4 inches, or 2.75 inches. It is noted that, in other embodiments, knob stack 124 may be slightly below knob stack 114, or at about the same level as that of knob stack 114, so long as the distance between knob stacks 114 and 124 in system 20 remains minimized relative to said distance in system 10.

Moreover, device 120 may be affixed to device 110, via fixture 210, at an angle preferred by the operator. This angle may refer to the angle between a plane on which knob stack 114 lies and a plane on which knob stack 124 lies. The angle may be any suitable angle that is ergonomic for operator use. For example, said angle may range from 0° to 100°, or 5° to 70°, but is not limited thereto. This angular orientation of knob stack 124, and thus device 120, is illustrated in the configurations shown in FIGS. 2A-2B. As can be seen in FIG. 2A, knob stack 114 lies on a plane that is parallel (or near parallel) to the page of FIG. 2A. Likewise, knob stack 124 lies on a plane that is parallel (or near parallel) to the page of FIG. 2A. Thus, in this configuration of system 20, knob stack 114 and knob stack 124 share the same plane or are in parallel planes, and the angle between the planes of knob stacks 114, 124 is essentially zero. In FIG. 2B, knob stack 114 lies on a plane that is parallel (or near parallel) to the page of FIG. 2B. However, knob stack 124 lies on a different plane that intersects the plane of knob stack 114, e.g., the page of FIG. 2B, at an angle, e.g., approximately 30°. Thus in this configuration of system 20, knob stack 124 (and device 120) may be at an angular orientation relative to knob stack 114 (and device 110).

In view of such positioning, as shown and described with reference to FIGS. 2A-2D, an operator may be able to access contact points, e.g., knob stacks 114 and 124 and ports 116 and 126, with minimal wrist rotation and/or forearm translation in a more stable position, relative to system 10. For example, an operator may access knob stacks 114 and 124 via wrist rotation or forearm rotation alone.

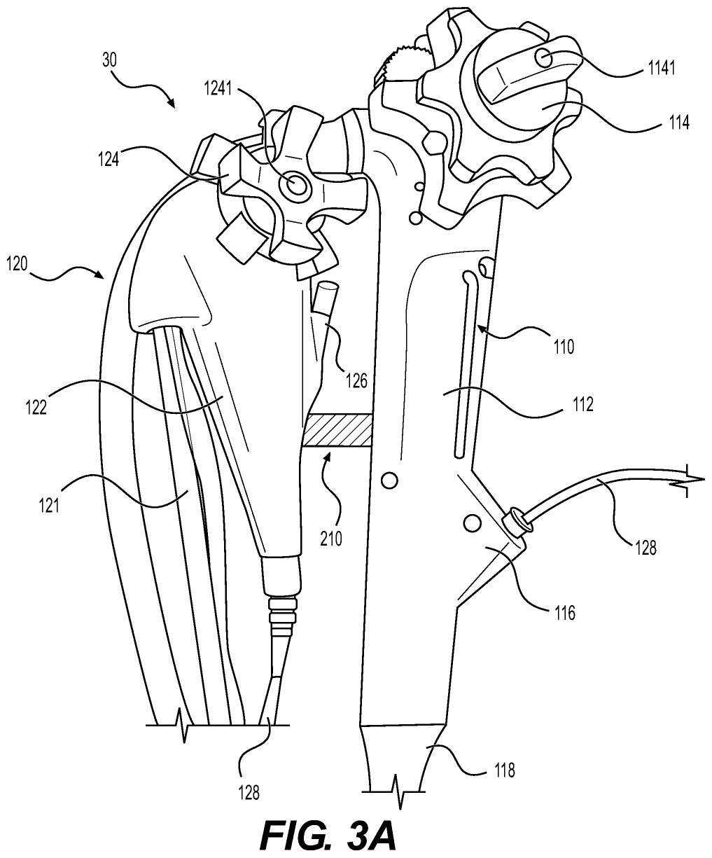

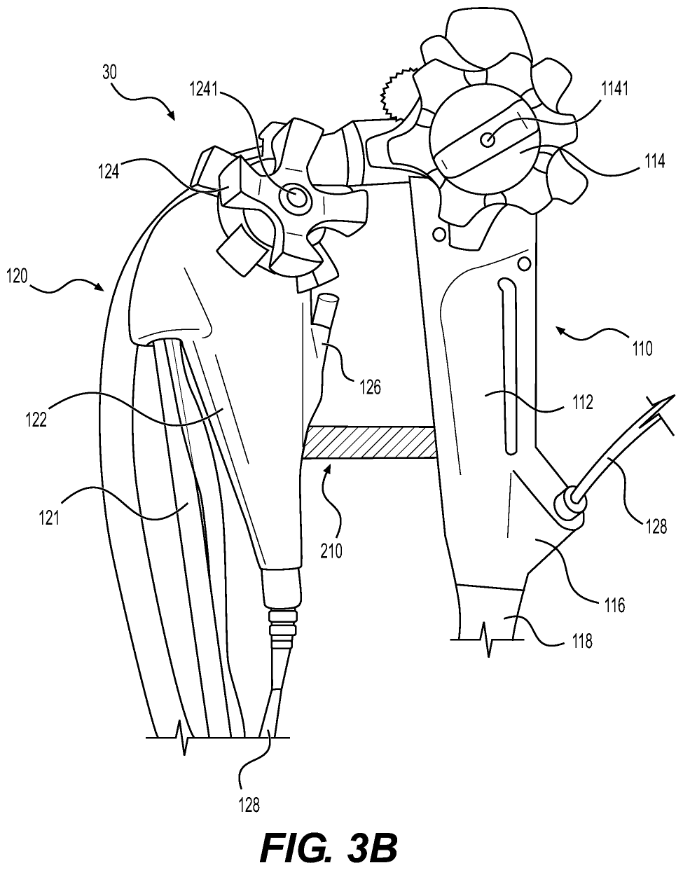

FIGS. 3A-3B illustrate another embodiment of a medical system 30. Like reference numerals refer to like parts. System 30 includes first medical device 110, second medical device 120, and a fixture 210. First medical device 110 and second medical device 120 may be the same devices as shown in systems 10, 20. Fixture 210, 210′, 210″, likewise, may be the same as fixture 210, 210′, 210″, as shown in system 20, configured to couple devices 110 and 120, and maintain both devices 110, 120 in the desired configuration. However, fixture 210, 210′, 210″ may be adjusted or oriented to accommodate for the configuration shown in FIGS. 3A-3B.

For example, referring to fixture 210′ (shown in FIG. 2C), to accommodate the positions of devices 110, 120 in FIG. 3A, flexible linkage 214′ may be adjusted to so that linkage 214′ extends behind handle 112 and second portion 216′ (and device 120) is closer to an operator than first portion 212′ (and device 110). Alternatively, fixture 210′ may be oriented so that second portion 216′ may be closer to an operator than first portion 212′, without adjusting or maneuvering linkage 214′, for example by manufacturing fixture 210′ in that fashion. In this orientation, second portion 216′ may receive the distal or lower portion of handle 122 of second device 120 via a channel/slot, and first portion 212′ may receive the distal or lower portion of handle 112 of first device 110 via a channel/slot. Said channel/slot of second portion 216′ may be configured to receive handle 122 at any suitable angular orientation of the plane of knob stack 124.

Referring to fixture 210″ (shown in FIG. 2D), to accommodate the positions of devices 110, 120 in FIG. 3A, flexible linkage 214″ may be adjusted so that linkage 214″ extends towards an operator so that second portion 216′ (and device 120) is closer to said operator than first portion 212′ (and device 110). Alternatively, fixture 210″ may be oriented so that second portion 216″ may be closer to an operator than first portion 212″, without adjusting or maneuvering linkage 214″, for example by manufacturing fixture 210″ in that fashion. In this orientation, second portion 216″ may receive the distal or lower portion of handle 122 of second device 120 via a channel/slot, and first portion 212″ may receive a portion of umbilicus 111 (or the strain relief of umbilicus 111) of first device 110. Said channel/slot of second portion 216″ may be configured to receive handle 122 at any suitable angular orientation of the plane of knob stack 124.

System 30 also yields improved ergonomics based on the positioning of first medical device 110 and second medical device 120, relative to one another. As shown in FIG. 3A, second medical device 120 may be closer to an operator (shown in FIG. 1), and first medical device 110 may be further from the operator relative to device 110. In system 30, an operator may hold the further first device 110 with one hand, e.g., the left hand, and may utilize the other hand, e.g., the right hand, to traverse between and operate aspects of devices 110 and 120, e.g., knob stacks 114 and 124. Second medical device 120 is also positioned such that knob stack 124 is slightly below knob stack 114. When system 30 is in use, and devices 110 and 120 are in the positions shown in FIG. 3A (handles 112 and 122 essentially upright/vertical), the degree by which knob stack 124 is below knob stack 114 may be, for example, from 0° to less than 90° degrees, or 5° to 30° degrees, between a horizontal line extending through center point 1141 of knob stack 114 and another line extending from point 1141 and center point 1241 of knob stack 124. Thus, the distance between knob stacks 114 and 124 in system 30 may also be minimized relative to said distance in system 10. In some examples, the distance between a center of knob stack 124 and a center of knob stack 114, in system 30, may be less than or equal to 6 inches, 4 inches, or 2.75 inches. It is noted that, in other embodiments, knob stack 124 may be slightly above knob stack 114, or at about the same level as that of knob 114, so long as the distance between knob stacks 114 and 124 in system 30 remains minimized relative to said distance in system 10.

Device 110 may be affixed to device 120, via fixture 210, at an angle, e.g., the angle between a plane on which knob stack 114 lies and a plane on which knob stack 124 lies, preferred by the operator. Said angle is not particularly limited, and may range from 0° to 100°, or 5° to 70° in certain examples. Examples of different angular orientations of device 120 are illustrated in the configurations shown in FIGS. 3A-3B. As can be seen in FIG. 3A, knob stack 114 and 124 lie on the same, or approximately the same plane. Thus, in this configuration of system 30, knob stack 114 and knob stack 124 share the same plane or are in parallel planes, and the angle between the planes of knob stacks 114, 124 is essentially zero. In FIG. 3B, knob stack 114 lies on a plane that is parallel (or near parallel) to the page of FIG. 3B. However, knob stack 124 lies on a different plane that intersects the plane of knob stack 114, e.g., the page of FIG. 3B, at an angle, e.g., approximately 30°. Thus in this configuration of system 30, knob stack 124 (and device 120) may be at an angular orientation relative to knob stack 114 (and device 110).

As shown and described with reference to FIGS. 3A-3B, the positioning of devices 110 and 120 in system 30 offers an operator the ability to maintain control between contact points. Moreover, an operator may access said contact points, e.g., knob stacks 114 and 124 and ports 116 and 126, with minimal wrist rotation and/or forearm translation in a more stable position, relative to system 10. This may contribute to decreased fatigue of an operator over the course of a procedure or multiple procedures.

It is noted that, due to the positioning of devices 110 and 120 in system 30, devices 110 and 120 may be structurally modified to accommodate for the device configurations. While not illustrated in FIGS. 3A-3B, for example, port 126 of device 120 may be situated on a position that is lower along handle 122 so that entry into port 126 is even less obstructed by knob stack 114 of device 110.

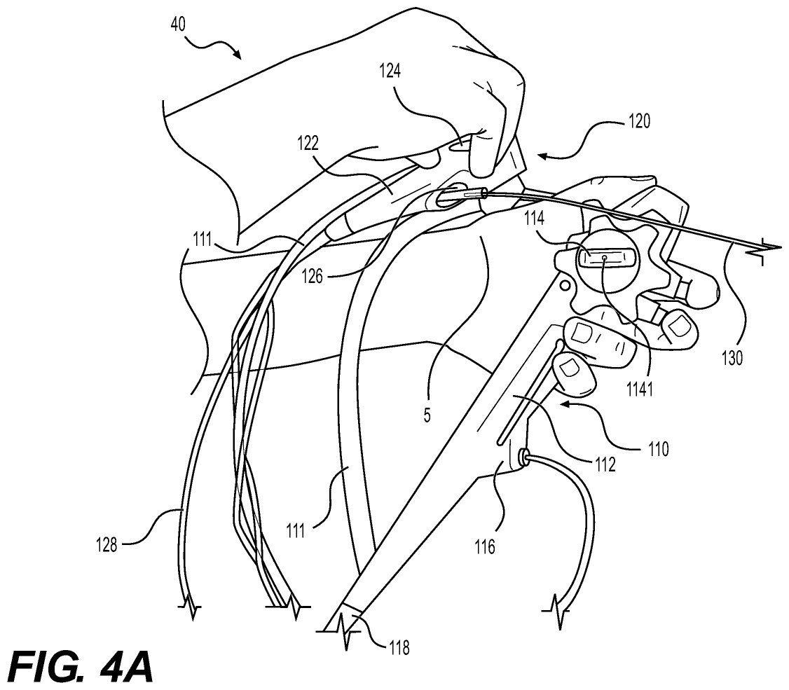

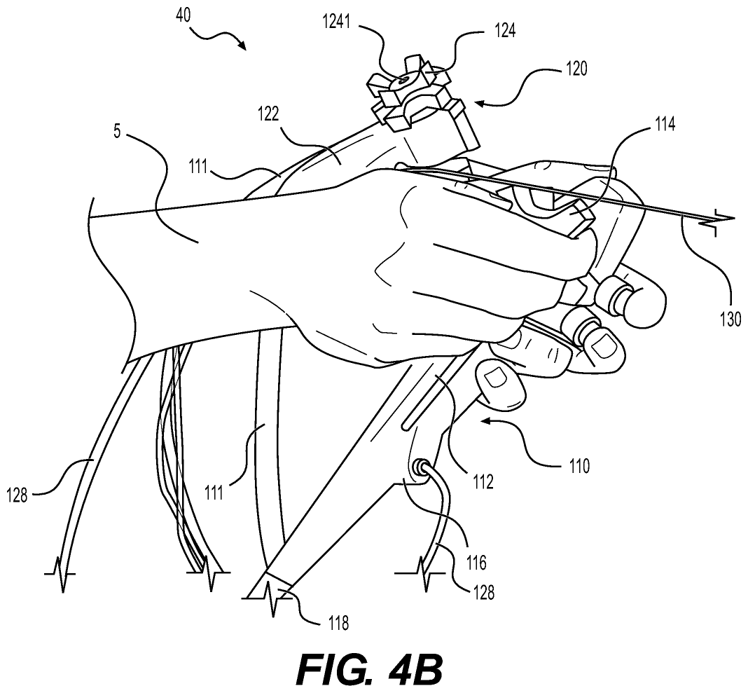

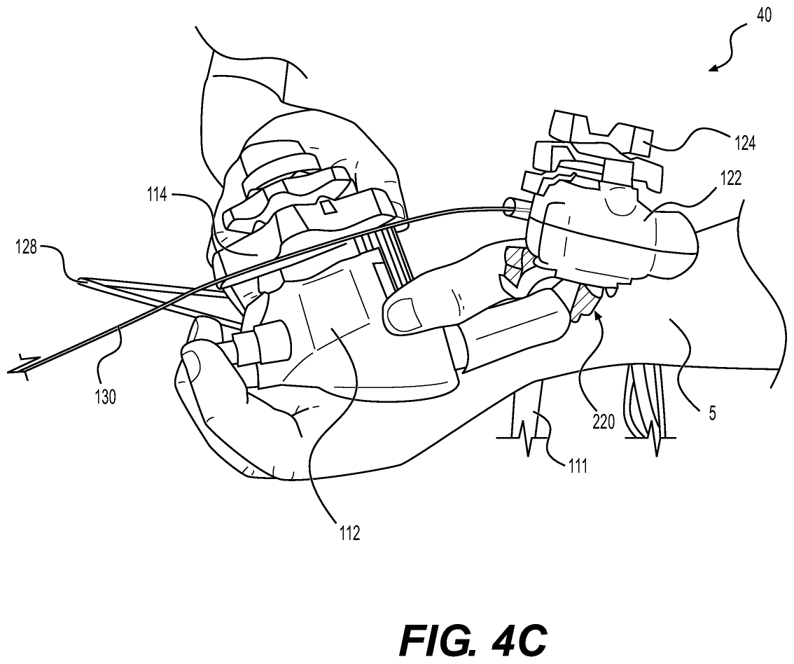

FIGS. 4A-4C illustrate another embodiment of a medical system 40. Like reference numerals refer to like parts. System 40 includes first medical device 110, second medical device 120, an accessory device 130, and a coupler 220 (see FIG. 4C). First medical device 110 and second medical device 120 may be the same devices as shown in systems 10, 20. Accessory device 130 is not particularly limited, and may be any suitable tool or device for medical purposes, e.g., forceps, needle, basket, etc. As shown, accessory device 130 may be introduced into port 126 of device 120, and may then extend distally through a lumen of shaft 128. Coupler 220 is not particularly limited, and may be any suitable coupling means, e.g., a clip, strap, clasp, adhesive, etc. configured to affix device 120 to umbilicus 111 or the strain relief of umbilicus 111. Coupler 220 may be removably or irremovably fixed to a portion of handle 122 or umbilicus 111 (or its strain relief). Moreover, coupler 220 may be adjustable so that a receiving portion of coupler 220 may adequately receive umbilicus 111 or its strain relief (if coupler 220 is affixed to handle 122) or receive handle 122 (if coupler 220 is affixed to umbilicus 111 or its strain relief). Coupler 220 may be secured onto handle 122 and umbilicus 111 or its strain relief via any suitable means.

In system 40, handle 112 of device 110 may be held by an operator via a hand 5, (such as a left hand as shown), and handle 122 of device 120 may be positioned so that it may rest against the hand 5 (and/or a portion of the wrist/forearm) of the operator. Handle 122 may rest against the hand 5 so that knob stack 124 and port 126 face in the general direction of the operator. Meanwhile, as shown in FIGS. 4A-4C, the other hand 6, (such as a right hand shown), may traverse between and operate aspects of devices 110 and 120, e.g., knob stacks 114, 124, ports 116, 126. In this configuration, the distance between knob stack 114 and knob stack 124 may be minimized relative to said distance in system 10, while also maintaining control between both knob stacks 114, 124. In some examples, the straight line distance from a center point 1241 (shown in FIG. 4B) of knob stack 124 to a center point 1141 (shown in FIG. 4A) of knob stack 114, in system 40, may be less than or equal to 7 inches, 5, inches, or 4.50 inches, and may depend on the size/length of the operator's hand and wrist. Thus, an operator may access said contact points, e.g., knob stacks 114 and 124 and ports 116 and 126, with minimal wrist rotation and/or forearm translation in a more stable position, relative to system 10.

Moreover, device 120 may be translatable along umbilicus 111 or rotated about umbilicus 111, while coupled via coupler 220, to a position that is most comfortable and ergonomic for an operator. An operator may also rotate the wrist of hand 5 to allow for minor adjustments in the angular relationship between the planes of knob stacks 114 and 124, to further improve the comfortability and ergonomics of system 40. It is further noted that, in system 40, port 126 and accessory device 130 may remain accessible at an angle similar to that of system 10. As shown in FIG. 4C, accessory device 130 may lie between knob stack 114 and handle 112, thereby minimizing any obstruction or interference with the handling of device 110, and also securing device 130 in a desired position.

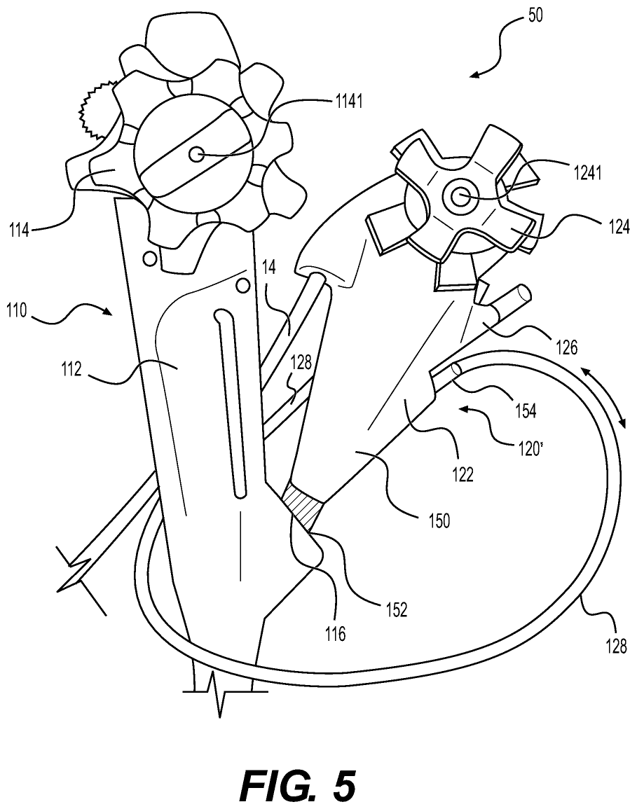

FIG. 5 illustrates another embodiment of a medical system 50. Like reference numerals refer to like parts. System 50 includes first medical device 110, and a second medical device 120′. First medical device 110 may be the same device as shown in any of the preceding Figures.

Second medical device 120′ may be structurally different than device 120 of the other systems discussed above. For example, in device 120′, shaft 128, which is in fluid communication with port 126, extends from a proximal end inside handle 122 at port 126, and through an opening (not shown in FIG. 5) on a side of handle 122 that is opposite of the side including knob stack 124, to exterior handle 122. Upon exiting that opening, shaft 128 may extend around a portion of device 110 and/or device 120′ in an at least partial loop, to then enter a port 154 of handle 122. Shaft then 128 extends within port 154, through an interior of handle 122 that is between port 154 and a receiving end 152 of handle 122. In this fashion, an operator may translate shaft 128 further in and out of port 154 (as indicated by the bi-directional arrow). Port 154 may be positioned on an upper portion of an anchor portion 150 of handle 122 that is accessible to an operator, so that an operator may adjust and maneuver shaft 128. For ergonomic reasons, procedural efficiency, etc., port 154 may be positioned along the same or similar plane as that of port 126. Port 154 may also extend radially outwards, relative to handle 122, at the same or similar angle as that of port 126. Thus, an operator may traverse between both ports 154 and 126 with minimal wrist and/or forearm rotation. A medical tool may be inserted in port 126 and thereby into shaft 128, ultimately to a treatment site.

Furthermore, a lower portion of handle 122 of second medical device 120′ may include anchor portion 150. Anchor portion 150 may be an aspect of device 120′ configured to receive or fix onto a portion of handle 112, thereby coupling devices 110 and 120′, as shown in FIG. 5. Anchor portion 150 includes at least one receiving end 152 at a lower end of portion 150. Receiving end 152 may be configured for receiving or coupling onto an aspect of device 110, e.g., port 116. Receiving end 152 is not particularly limited and may be, for example, an opening, channel, etc. Receiving end 152 may receive or couple onto device 110 via any suitable means, e.g., clamping, frictional fit, adhesion, etc. Anchor portion 150 may be internally structured so that port 154 and receiving end 152 are in fluid communication with the entrance of port 116 (shown in FIG. 1) of device 110. Thus, device 120′ may be anchored to device 110 via receiving end 152, and shaft 128 may extend through port 154 of device 120′ and through port 116 of device 110.

It is noted that in some embodiments, anchor portion 150 may be a separate component that is removably coupled to a portion of handle 122. Anchor portion 150 may be removably coupled to a portion of handle 122, e.g., a lower portion, via any suitable coupling feature (not shown), e.g., frictional fit, snap-on, etc. Furthermore, in other embodiments, anchor 150 portion may be an integrated feature or a removable feature/component of handle 110 that is configured to receive a portion of handle 122 and insertion tube 128. In such embodiments, device 110 and/or device 120′ may be structurally modified or altered to accommodate for the integration of anchor portion 150 or for the coupling between devices 110, 120′.

System 50 yields improved ergonomics based on the positioning of second medical device 120′ relative to first medical device 110. As a result of the anchoring shown in FIG. 5, first medical device 110 may be closer to an operator (shown in FIG. 1), and second medical device 120′ may be further from said operator relative to first medical device 110. In system 50, an operator may hold the closer, first device 110 with one hand, such as the left hand, and may utilize the other hand, such as the right, to traverse between both devices 110, 120′ and operate the contact points of devices 110, 120′, e.g., knob stacks 114 and 124. The distance between an operator and second device 120′ is not particularly limited, and may depend on the position of anchor portion 150 with respect to handle 112. Second medical device 120′ is also positioned such that knob stack 124 is slightly below knob stack 114, although not limited thereto. Moreover, device 120′ may be anchored to device 110, via anchor portion 150, at an angle, depending on the angle of receiving end 152. It is noted that said angle is not particularly limited, and may be any suitable angle that maintains the ergonomics of system 50.

It is further noted that device 120′ may rotate to alter the angle between a plane on which knob stack 114 lies and a plane on which knob stack 124 lies. For example, receiving end 152 may be coupled onto port 116 of device 110, e.g., via a frictional fit, thereby allowing some angular rotation of device 120′ relative to device 110. The angle may be any suitable angle that is ergonomic for operator use. For example, said angle may range from 0° to 100°, or 5° to 70°, but is not limited thereto. As can be seen in FIG. 5, knob stack 114 lies on a plane that is parallel (or near parallel) to the page of FIG. 5. Likewise, knob stack 124 lies on a plane that is parallel (or near parallel) to the page of FIG. 5. Thus, in this configuration of system 50, knob stack 114 and knob stack 124 share the same plane or are in parallel planes, and the angle between the planes of knob stacks 114, 124 is essentially zero. However, in other examples of system 50, the angle may be adjusted, via rotation of device 120′, so that the angle is, for example, approximately 20°, 30°, or 40°.

In view of such positioning, as shown and described with reference to FIG. 5, an operator may be able to access contact points, e.g., knob stacks 114 and 124 and ports 116, 126, and 154, with less wrist rotation and/or forearm translation in a more stable position, relative to system 10.

It will be apparent to those skilled in the art that various modifications and variations can be made to the disclosed device without departing from the scope of the disclosure. Other embodiments of the disclosure will be apparent to those skilled in the art from consideration of the specification and practice of the invention disclosed herein. It is intended that the specification and examples be considered as exemplary only, with a true scope and spirit of the invention being indicated by the following claims.

What is claimed is:

1. A medical system, comprising:
a first device comprising a first handle and a first shaft extending distally from the first handle, wherein the first handle includes a first actuator;
a second device comprising a second handle and a second shaft extending distally from the second handle, wherein the second handle includes a second actuator; and
a fixture coupling the first device to the second device,
wherein a center of the first actuator is within 4.5 inches of a center of the second actuator, and
wherein the fixture couples the first device to the second device so that the first handle is configured to be held in a first hand of an operator, while a second hand of the operator traverses between use of the first actuator and the second actuator.

2. The medical system of claim 1, wherein, during use, the second shaft enters a port of a handle of the first device, the first device is configured to be closer to the operator than the second device, and the first actuator is configured to be below the second actuator.

3. The medical system of claim 1, wherein, during use, the second shaft enters a port of a handle of the first device, the first device is configured to be further from the operator than the second device, and the first actuator is configured to be above the second actuator.

4. The medical system of claim 1, wherein the first actuator lies on a first plane, the second actuator lies on a second plane, and the second plane is at an angle relative to said first plane, wherein the angle between the second plane and the first plane is between 5° and 70°.

5. The medical system of claim 1, wherein the first actuator and the second actuator lie on a shared plane or parallel planes.

6. The medical system of claim 1, wherein the first actuator includes a first rotatable knob, the second actuator includes a second rotatable knob, and an axis of rotation of the first rotatable knob is within 2.75 inches of an axis of rotation of the second rotatable knob.

7. The medical system of claim 1, wherein the coupling fixture comprises a first section receiving the first device and a second section receiving the second device, wherein the second section is configured to receive the second device at any angular orientation of the second device relative to the first device.

8. The medical system of claim 7, wherein the first section receives a portion of the first handle of the first device, and the second section receives a portion of the second handle of the second device.

9. The medical system of claim 8, wherein the first section includes a first channel configured to receive the portion of the first handle, the second section includes a second channel configured to receive the portion of the second handle, and the coupling fixture further includes an arm extending between the first section and the second section.

10. The medical system of claim 7, wherein the first device further comprises a connector cable extending proximally, wherein the first section receives a portion of said connector cable, and the second section receives a portion of the second handle of the second device.

11. The medical system of claim 7, wherein the coupling fixture further comprises an adjustable arm extending between the first section and the second section, wherein the adjustable arm is configured to be maneuverable so that the second section is at any angular orientation relative the first section.

12. The medical system of claim 1, wherein the first device further comprises a first port, the second device further comprises a second port and a third port, and wherein a portion of the second device is coupled to the first port.

13. The medical system of claim 12, wherein the second shaft of the second device is configured to extend from a proximal end at the second port to the third port, and the third port of the second device is in fluid communication with the first port of the first device.

14. The medical system of claim 1, wherein the second device is configured to be oriented relative to the first device so that the second device can rest again the first hand when the first hand holds the first handle of the first device.

15. The medical system of claim 1, wherein the second device is configured to be oriented relative to the first device so that the operator can access the first actuator and the second actuator via only forearm and/or wrist rotation.

16. A method of handling a medical system including a first device and a second device, comprising:
orienting the medical system so that the first device is closer to an operator than the second device;
holding a handle of the first device with a first hand of the operator; and
traversing between an actuator of the first device and an actuator of the second device with a second hand, via only wrist and/or forearm rotation of a second hand of the operator.

17. The method of claim 16, further comprising adjusting an angle of a second plane on which the second actuator lies relative to a first plane on which the first actuator lies, wherein the angle ranges from 5° to 70°.

18. The method of claim 17, wherein the medical system further comprises a coupling fixture coupling the first device and the second device, and the angle is adjusted via adjusting the coupling fixture.

19. The method of claim 16, wherein the actuator is a first rotatable knob, the second actuator is a second rotatable knob, and an axis of rotation of the first rotatable knob is within 2.75 inches of an axis of rotation of the second rotatable knob.

20. A medical system, comprising:
a first device comprising a first handle and a first shaft extending distally from the first handle, wherein the first handle includes a first actuator;
a second device comprising a second handle and a second shaft extending distally from the second handle, wherein the second handle includes a second actuator; and
a fixture coupling the first device to the second device, wherein the fixture couples the first device to the second device so that the first handle is configured to be held in a first hand of an operator, while a second hand of the operator traverses between use of the first actuator and the second actuator, and
wherein a position of the second handle relative to a position the first handle is adjustable via the fixture to minimize the traversal of the second hand between use of the first actuator and the second actuator.
